(19) United States
(12) Patent Application Publication
(10) Pub. No.: US 2003/0109827 A1
(43) Pub. Date: Jun. 12, 2003

(54) Drug delivery system and method

(75) Inventors: Gilad Lavi, Rishon Lezion (IL); Gil Ygal, Gan-Yavne (IL); Israil Tsals, Newtown, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(73) Assignee: ELAN PHARMA INTERNATIONAL LIMITED, Shannon County Clare (IE)

(21) Appl. No.: 10/010,624

(22) Filed: Dec. 7, 2001

(51) Int. Cl. A61M 5/20; A61M 5/00

(52) U.S. Cl. 604/134

(57) ABSTRACT

A system and method for delivering drug solutions to a user, the system having a housing with a drug reservoir therein. The system also includes an activation assembly having a delivery needle and an activation device for activating delivery. The system further includes a pump, associated with the drug reservoir and a flow restrictor. When activated, the pump moves the drug out of the drug reservoir through the delivery needle and into the skin of the user. The pump may be in the form of a spring.

### BACKGROUND OF THE INVENTION

The present invention relates to the administration of a drug solution and, more particularly, to the delivery of a viscous drug solution into a mammal.

Various devices have been developed for the delivery of medications into living organisms, including syringes in which a liquid is delivered from a chamber using pressure asserted by a manual plunger through a needle inserted under the skin.

However, certain drug solutions, in their deliverable state are highly viscous. Furthermore, the drug volumes frequently exceed 1 milliliter (ml), a volume that could be effectively absorbed by the subcutanceous tissue upon injection. These include certain drugs delivered subcutaneously to enhance fertility, treat cancer, rheumatoid arthritis and multiple sclerosis, allergies, and other conditions. For example, one such cited compound has a viscosity of about 50 cp (centapoise). The delivery of these types of drugs presents certain challenges as delivery of such solutions by means of a typical syringe is inappropriate and may be ineffective. Highly viscous drugs are more difficult to deliver because they require more pressure to deliver using standard technology and also need a longer period of time to absorb into the system of the patient. Highly viscous drugs are difficult to inject by hand because it requires more pressure on the syringe plunger to move drug out of the syringe body through the needle and into the user. The patient may be unable to provide such pressure for an extended period of time, which results in a incomplete dosage, or the patient may move the needle when applying the additional pressure during delivery resulting in unnecessary infliction of pain to the patient.

In addition to highly vicous drugs, it should be mentioned that high dosage volumes of low viscous formulations, such as water, also do not get absorbed if injected using existing technology. An injection of a high volume dosage using existing technology, such as a standard syringe, will result in intolerable pain to the patient. This is due to the body's inability to quickly absorb such a volume of drug in a short period of time. Thus, it is necessary for high volume doses to be delivered at a rate at which the body can absorb the drug solution. As a result, the delivery time needs to be longer. Using standard technology, the injection of a high volume dosage would likely result in movement of the needle during delivery which inflicts unneeded pain on the patient and may result in an incomplete delivery.

Other drug delivery devices have been designed to delivery drug solution. These include applicants' own inventions disclosed and described in U.S. Pat. Nos. 5,527,288; 5,997,501; 5,848,991; 5,814,020; and 5,858,001; as well as U.S. patent application Ser. Nos. 09/072,875; and 09/439,879. These inventions are directed to various drug delivery systems. However, such devices and methods are not appropriate for delivering viscous drug solutions as they require considerable more energy to move the drug out of the drug reservoir and into the user. Moreover, the present invention requires considerable time for delivery due to the user's inability to quickly absorb the drug solution into his/her system. The combination of increased power requirements combined with increased delivery time for injection would make modification of the aforementioned inventions impracticable and ineffective.

Thus, there is a need for a drug delivery device for the delivery of viscous drug solutions.

There is a further need for a drug delivery device for the delivery of viscous drug solutions that effectively delivers the appropriate volume of drug to the user in a manner that enables the drug solution to become properly absorbed into the user's system during delivery without inflicting any unnecessary pain.

There is yet a further need for a drug delivery device for the delivery of high volume dosage of drug solution capable of delivering the volume at a rate at which the body can absorb the drug so that the dosage is effective and pain during delivery to the patient is minimized.

### SUMMARY OF THE INVENTION

The present invention relates to systems and methods for delivering drug solutions to a user over an extended period of time. The system includes a housing and a drug reservoir housed in the housing. The system also includes an activation assembly having a delivery needle for insertion into the skin of a user, and an activation device for activating liquid communication between the drug reservoir and delivery needle and for activating the insertion of the delivery needle into the skin. The system also includes a movable channel that effects liquid communication between the drug reservoir and the delivery needle upon activation. A flow restrictor is in communication with the moveable channel to restrict the flow of drug therethrough. The system further includes a pump for moving drug out of the drug reservoir, through the delivery needle and into the user when activated.

In a preferred embodiment, the pump is a spring, biased against the drug reservoir. When activated, the spring forces drug out of the drug reservoir through the flow restrictor and needle and into the user.

In a preferred embodiment, the flow restrictor is a length of tubing. The preferred range of diameter of tubing is from 0.5 to 0.05 mm and the preferred length of tubing can go up to about 10 cm. In a preferred embodiment, the delivery rate of drug using the system has been tested to achieve flow rates of between 2 and 3 ml/hour.

In a preferred embodiment, the activation device is a knob. When the knob is rotated, it results in liquid communication between the drug reservoir and delivery needle, and when it is subsequently depressed, it results in movement of the delivery needle into the skin to active delivery.

The system also includes the ability to move the delivery needle from a first storage position, to a second delivery position to a third locked position. This enables the system to be safely stored prior to activation and to prevent reuse or contamination or harm to the user or health care worker upon completion of use of the system.

The system may also include a layer of adhesive on the housing that contacts the skin during delivery. The adhesive acts to secure the housing, and more particularly the needle, during delivery.

The foregoing and other objects, features, and advantages of the drug delivery systems and methods will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### BRIEF DESCRIPTION OF THE DRAWINGS

FIG. 1 is a front cross-sectional view of the preferred embodiment of the drug delivery system of the present invention prior to use;

FIG. 1A is a top sectional view of the embodiment of FIG. 1;

FIG. 1B is a side sectional view of the of the embodiment of FIG. 1;

FIG. 1C is a side view of an alternative embodiment of FIG. 1 having an adhesive layer;

FIG. 1D is a front view of the embodiment of FIG. 1C;

FIG. 2 is a front cross-sectional view of the preferred embodiment of the drug delivery system of FIG. 1 initiating use;

FIG. 2A is a top sectional view of the embodiment of FIG. 2;

FIG. 2B is a side sectional view of the of the embodiment of FIG. 2;

FIG. 3 is a front cross-sectional view of the preferred embodiment of the drug delivery system of the present invention during delivery of the drug solution;

FIG. 3A is a front cross-sectional view of the preferred embodiment of the drug delivery system of the present invention at the end of delivery of the drug solution;

FIG. 3B is a top sectional view of the embodiment of FIG. 3A;

FIG. 3C is a side sectional view of the of the embodiment of FIG. 3B;

FIG. 4 is a front cross-sectional view of the preferred embodiment of the drug delivery system of the present invention after use;

FIG. 4A is a top sectional view of the embodiment of FIG. 4;

FIG. 4B is a side sectional view of the embodiment of FIG. 4;

FIG. 4C is a front cross-sectional view of the preferred embodiment of the drug delivery system of the present invention in its final and locked position;

FIG. 4D is a side sectional view of the embodiment of FIG. 4C;

FIG. 4E is a top sectional view of the embodiment of FIG. 4C;

FIG. 5 is a perspective view of the drug reservoir and needle assembly prior to use;

FIG. 6 is a second perspective view of the embodiment of FIG. 5;

FIG. 7 is a perspective view of the drug reservoir and needle assembly initiating use;

FIG. 8 is second perspective view of the embodiment of FIG. 7;

FIG. 9 is a perspective view of the drug reservoir and needle assembly during drug delivery;

FIG. 10 is second perspective view of the embodiment of FIG. 9;

FIG. 11 is a perspective view of the drug reservoir and needle assembly after use;

FIG. 11A is a second perspective view of the embodiment of FIG. 11;

FIG. 12 is a perspective view of the drug reservoir and needle assembly during in its final and locked position;

FIG. 14 is a second perspective view of the embodiment of FIG. 13;

FIG. 15 is a perspective view of the embodiment of FIG. 1;

FIG. 16 is a schematic view of the set up for experiments using the present invention;

FIG. 17 is a table illustrating the results of an experiment using a 40 mm tube length with a 0.05 mm diameter at a starting pressure of about 950 mbar;

FIG. 18 is a table illustrating the results of an experiment using a 140 mm tube length with a 0.05 mm diameter at a starting pressure of about 1000 mbar;

FIG. 19 is a table illustrating the results of an experiment using a 70 mm tube length with a 0.05 mm diameter at a starting pressure of about 950 mbar;

FIG. 20 is a table illustrating the results of an experiment using a 70 mm tube length with a 0.05 mm diameter at a starting pressure of about 675 mbar; FIG. 21 is a table illustrating the results of an experiment using a 70 mm tube length with a 0.2 mm diameter at a starting pressure of about 400 mbar;

FIG. 22 is a table illustrating the results of an experiment using a 140 mm tube length with a 0.2 mm diameter at a starting pressure of about 650 mbar;

FIG. 23 is a table illustrating the results of an experiment using a 70 mm tube length with a 0.05 mm diameter at a starting pressure of about 900 mbar; and

FIG. 24 is a table illustrating the results of an experiment using a 70 mm tube length with a 0.05 mm diameter at a starting pressure of about 650 mbar.

### DETAILED DESCRIPTION OF THE INVENTION

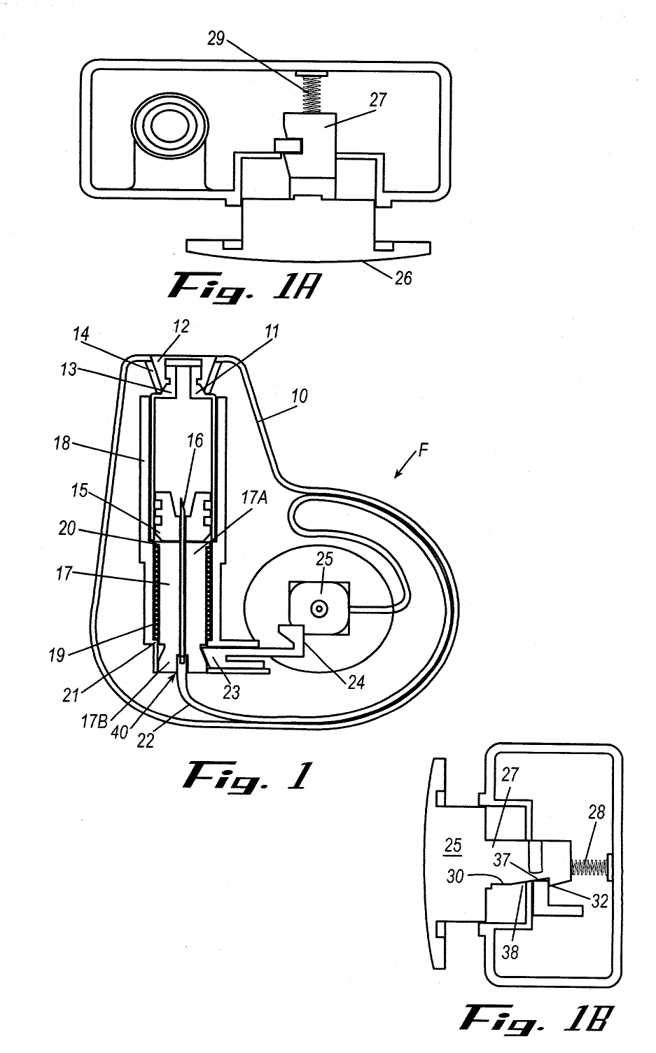

Referring now in more detail to the drawing, which illustrates the general structure and function of preferred embodiments of the invention, a preferred embodiment or device 5 includes a housing 10, as shown in FIGS. 1A-C. The housing 10 also includes a drug cartridge 11 which is inserted through an opening 12 in the housing. The cartridge 11 has a neck 13 which is held in place by a conical depression 14 in the housing 10 when the cartridge is fully inserted into the device 5. The cartridge 11 further includes a rubber plunger 15. When the cartridge 11 is inserted into the opening 12 in the housing 10, the plunger 15 is pierced by an internal needle 16. The internal needle 16 is held within a cylinder 17. The cylinder 17 is held within a recess 18 within the housing. The cylinder 17 is pre-loaded by means of spring 19 placed along the outer axis 17A of the cylinder. The spring 19 is held within a portion of the length of the recess 18 by cylinder lip 20 and recessed step 21. At the bottom end 17B of the cylinder 17 is a central opening 40 through which the needle 16 projects.

The needle 16 is connected to a length of tubing 22. The tubing acts as a flow restrictor and limits the flow rate of the drug solution as it travels through the length of tubing. The cylinder 17 has an angled base for matingly fitting with a latch 23. The latch has a head 24 for engagement with a portion of an injector knob 25. As can be seen from the top view, FIG. 1(A), the knob 25 has an outer face 26 and a central axis 27 which is fixed to a delivery needle 28 which is spring loaded within the housing 10 by means of an injection coil spring 29.

The housing may include an adhesive layer 60 which is applied to the surface of the housing 10 that is in contact with the user's skin during delivery, as shown in FIGS. 1C and 1D. The adhesive layer 60 is protected prior to use by a removable liner 62, typically made of paper or plastic. The liner 62 has a tab 64 protruding from one portion of the housing 10 to ease in grabbing and removing the liner 62.

To use the device, the cartridge 11 is inserted into the opening 12 within the housing 10. The cartridge 11 is depressed into the opening 12 until the neck of the cartridge is flush against the outer surface of the housing 10, as shown in FIG. 1A. As the cartridge 11 is depressed, the needle 16 is pressed against the plunger 15 and penetrates through the plunger to establish liquid communication with the drug solution in the cartridge. At this point, the drug is ready for injection into the user.

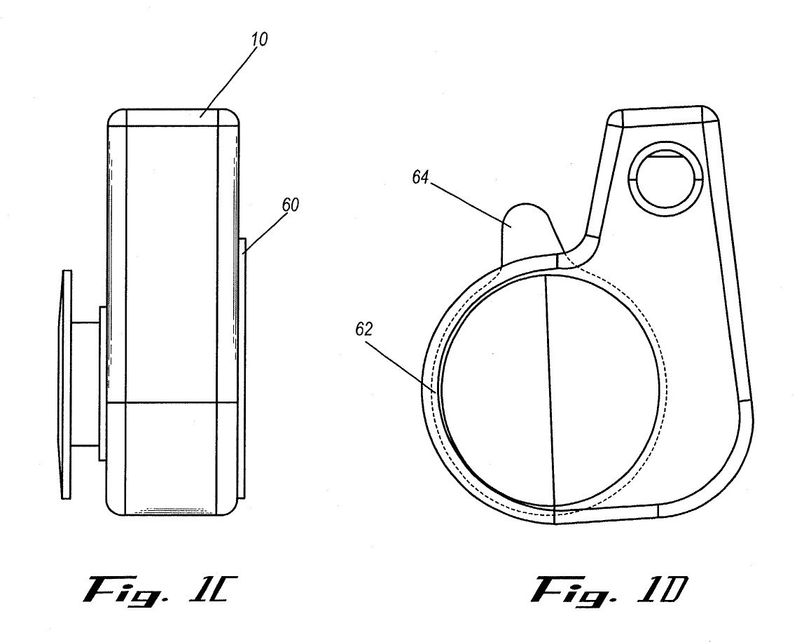

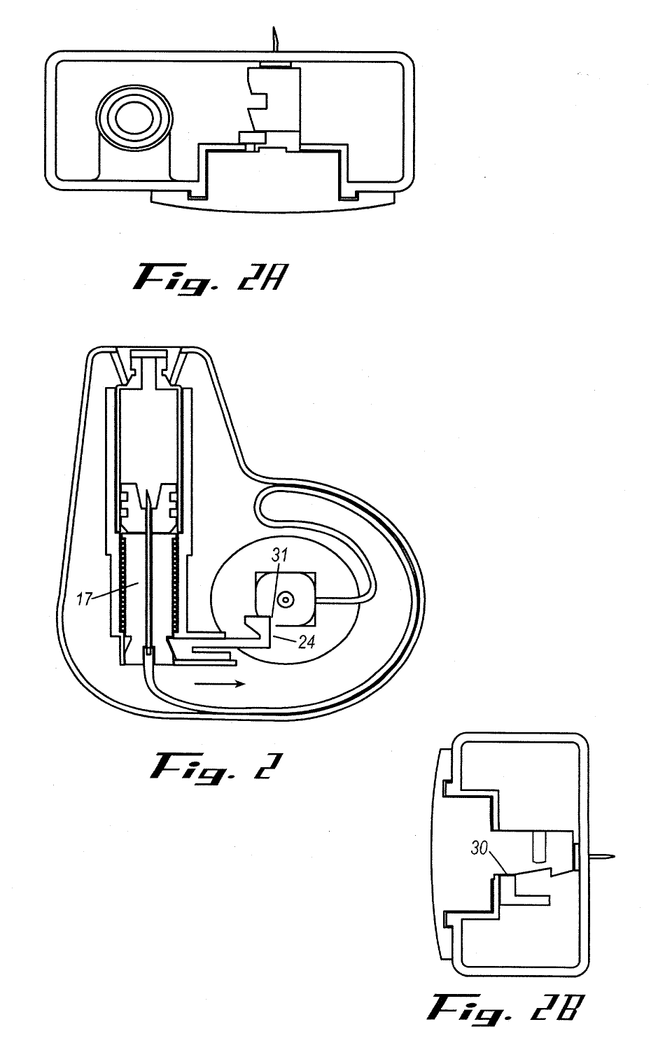

Prior to use, the user peels off the removable liner 62 from the adhesive layer 60 to expose the adhesive. The user places the housing 10 against the skin at the injection site so that the adhesive 60 is in contact with the skin and secures the housing thereto. Then, the user depresses the knob 25 so that the face of the knob 26 becomes flush with the housing 10 as shown in FIG. 2. The depression of the knob 25 causes the latch head 24 to move from a first angled recess 35 in the knob axis to a second recess 30. This movement causes the head 24 to clear the mating engagement of the protuberance on the head 31 with the mating recess in the shaft 27 of the knob 25 as shown in FIG. 2(B). As the protuberance on the latch head 31 clears the knob shaft recess 35, the angled base of the cylinder 17 causes the latch 23 to move in a lateral direction to the right as indicated by the arrow in FIG. 2. This results in a clearance of the latch 23 from the cylinder base 17B which enables the cylinder 17 to travel upward under the force of the spring 19. As the cylinder 17 moves upward it causes the drug within the cartridge 11 to move out of the cartridge through the tubing 22 and into the delivery needle 28, as shown in detail in FIG. 3A. Concurrently, depression of the knob 25 also causes the delivery needle 28 to move out of the housing 10 and into the skin of the user. The latch head 23 is received into the second recess 30 during use. The angled slope 37 of the first recess 35 allows it to move into the second recess 30 and the vertical surface 38 within the housing prevents the delivery needle 28 from retracting back into the housing 10 during use.

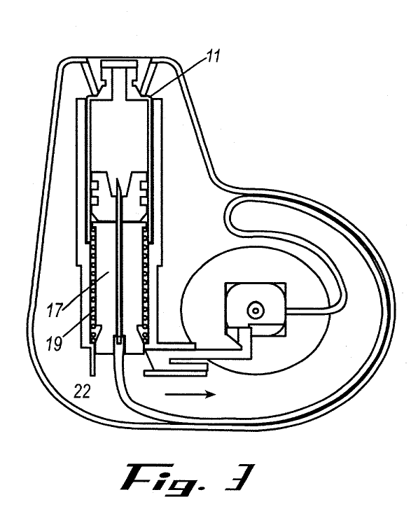

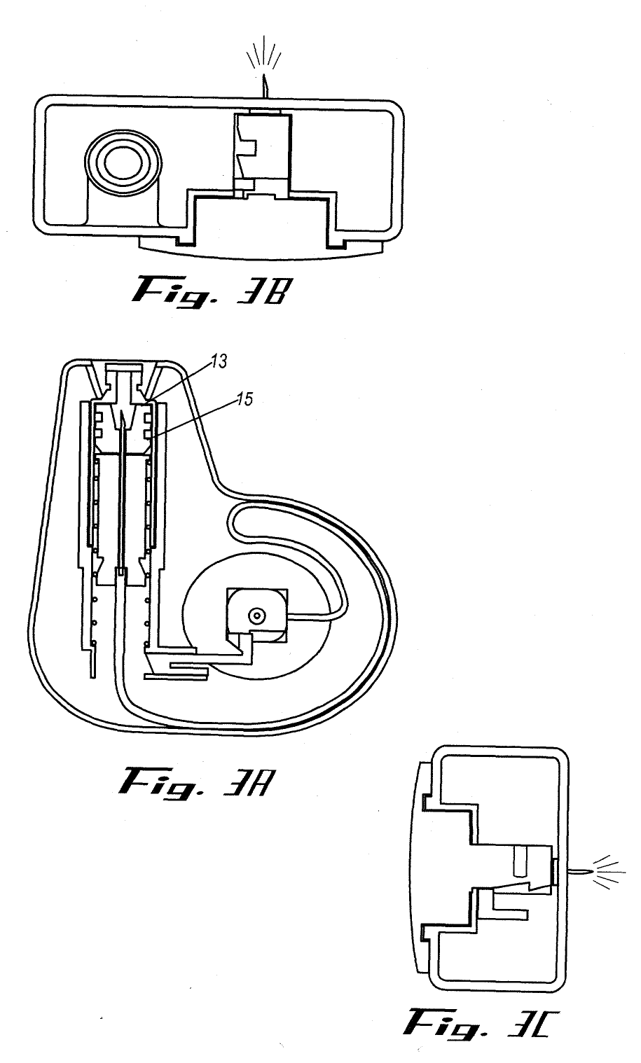

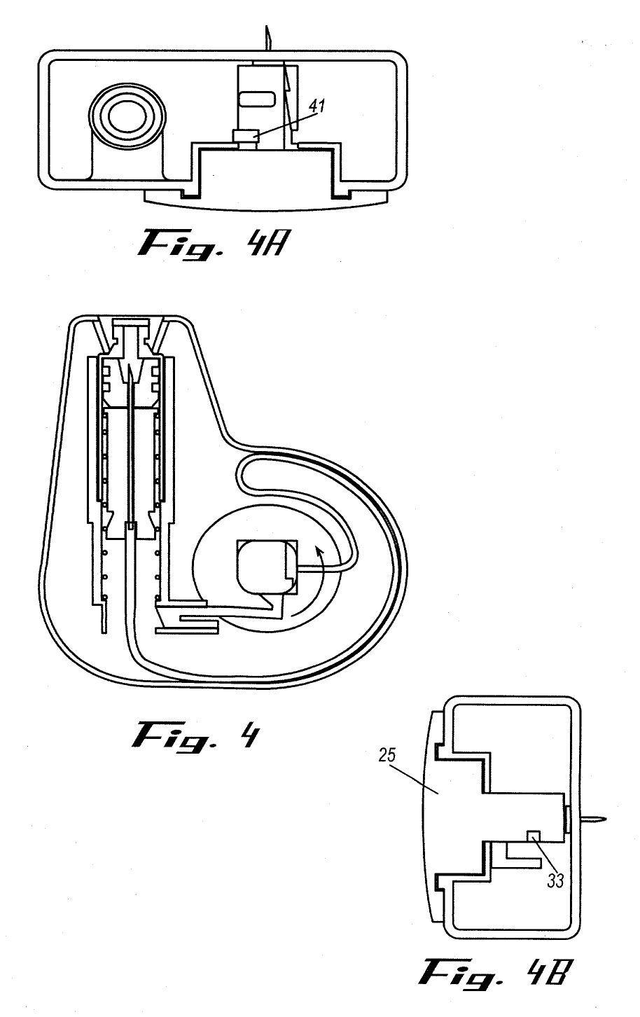

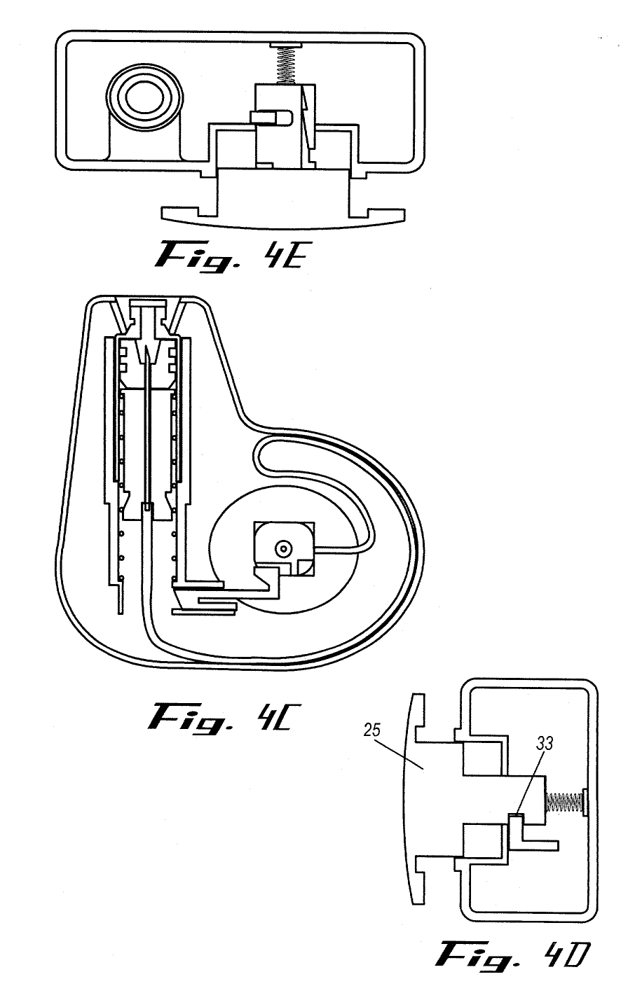

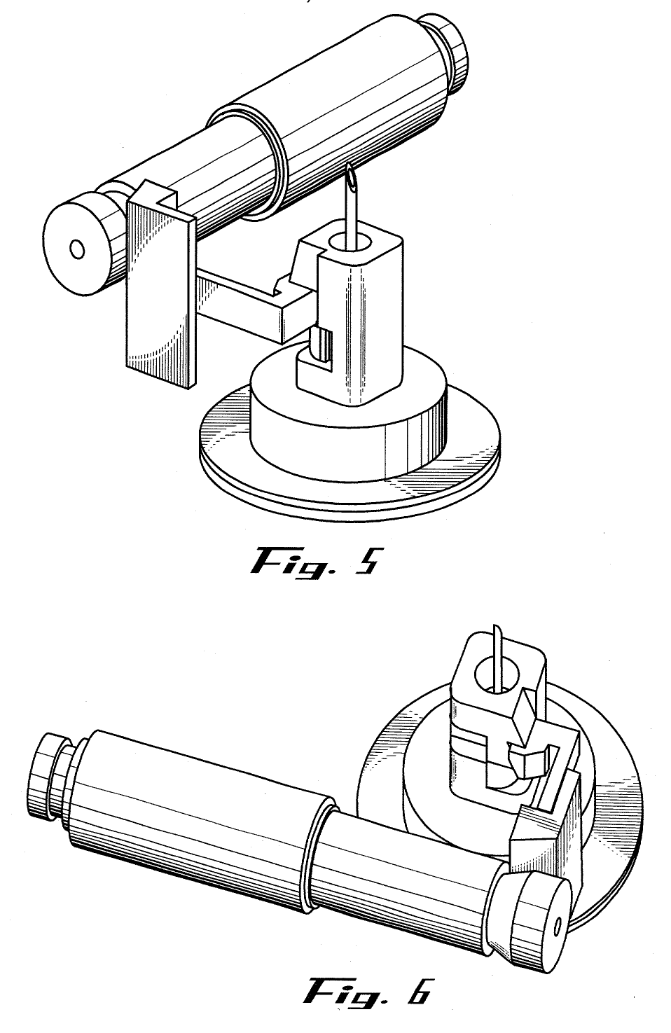

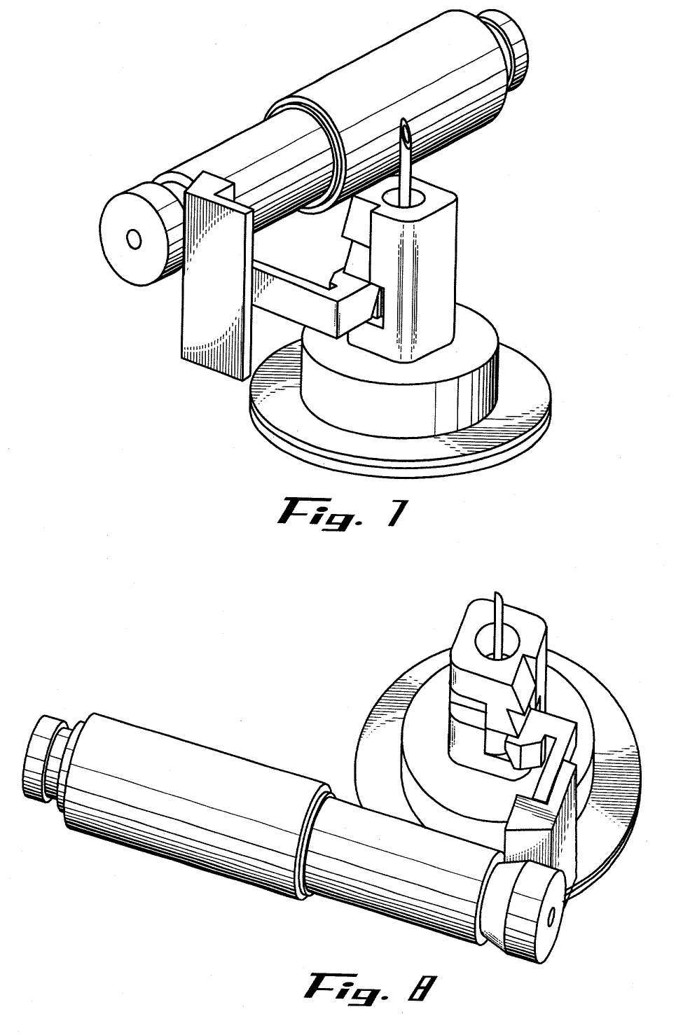

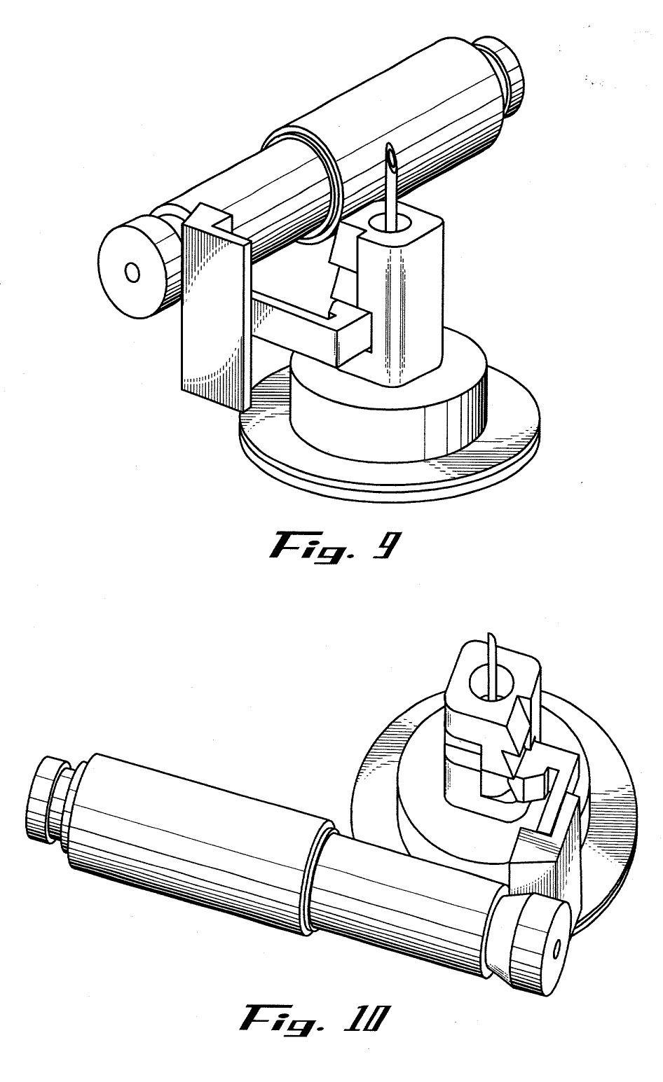

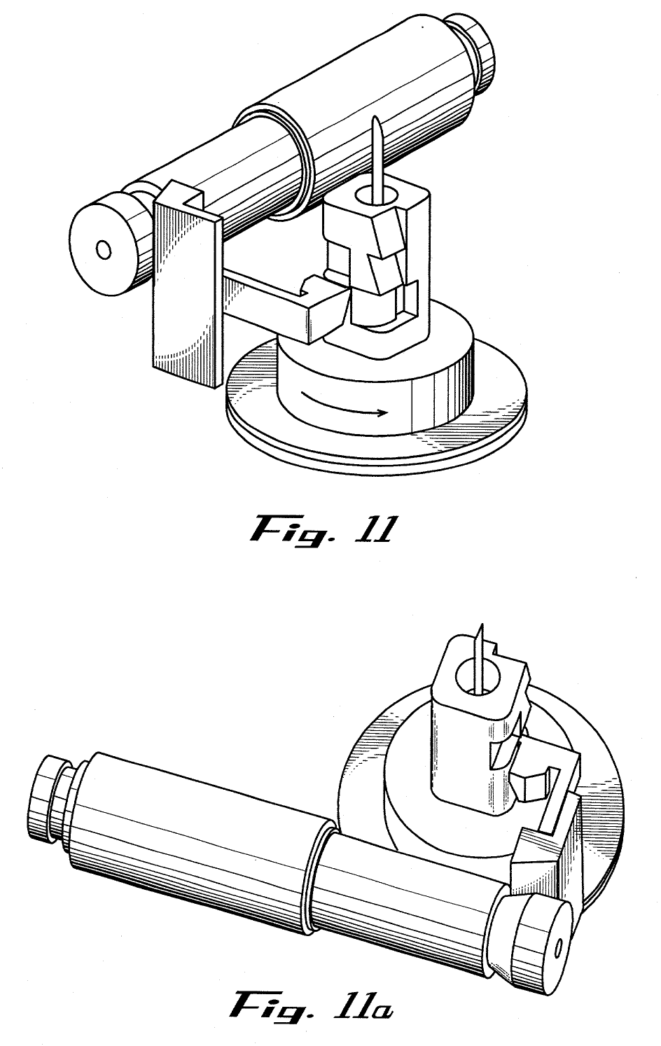

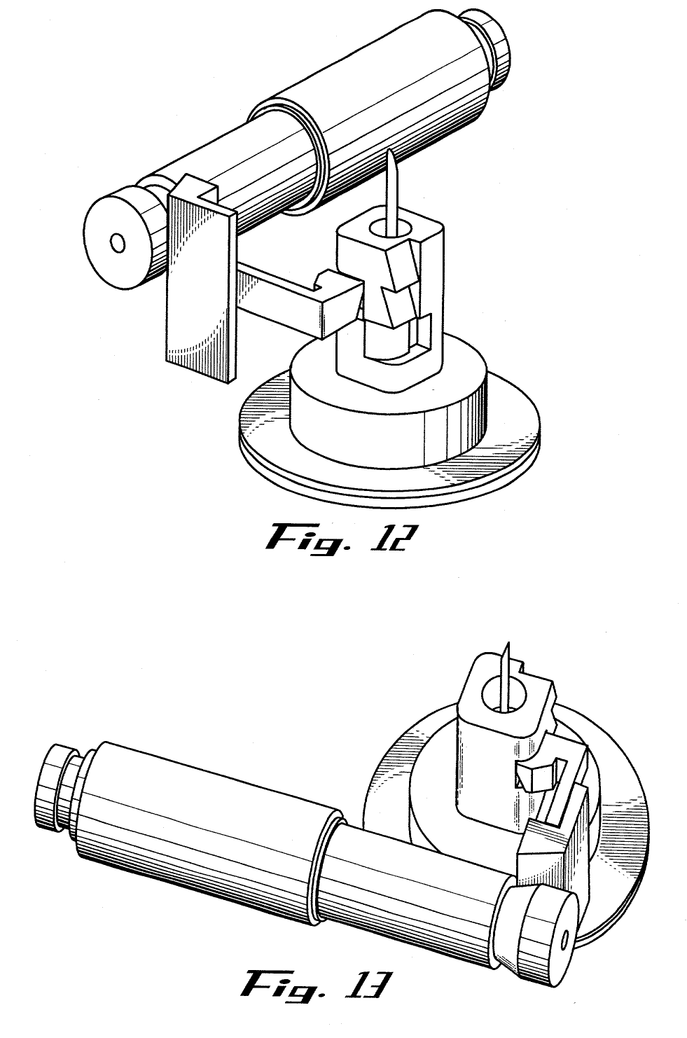

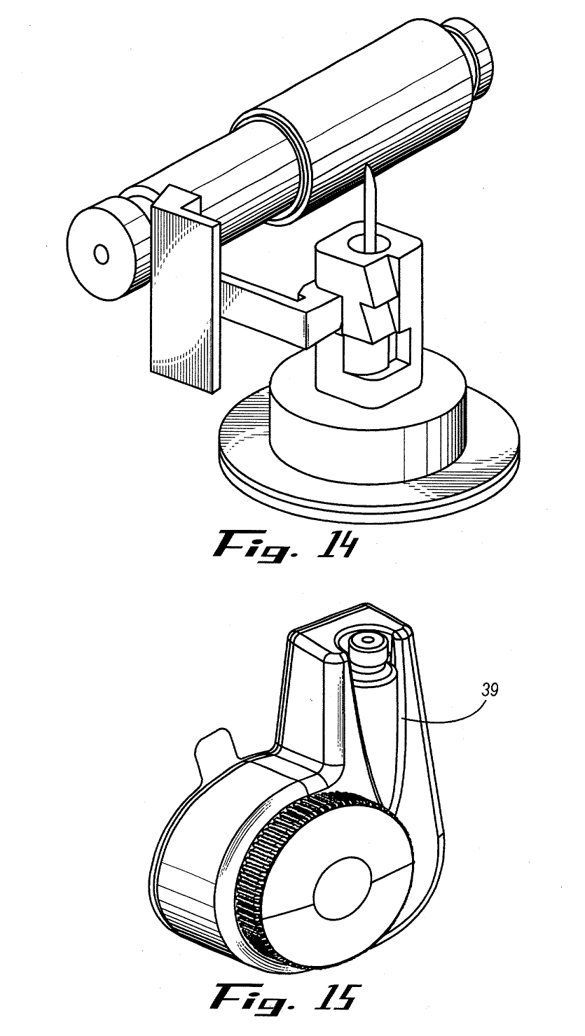

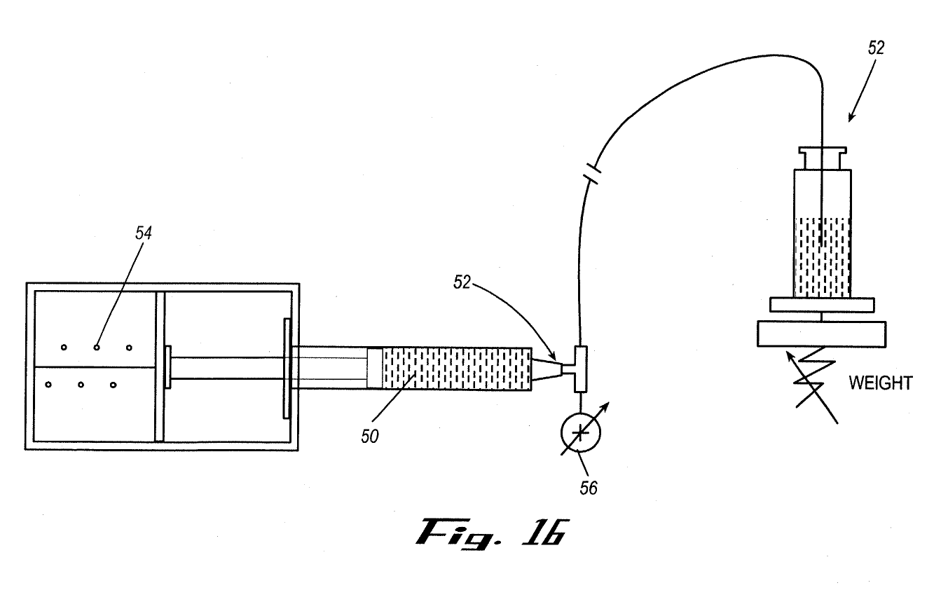

Once the delivery is complete, the stopper 15 will end its travel along the length of the cartridge 11 and rest on the cartridge lip 13. At that time, the user will be able to view the end of delivery indication through a slot 39 within the housing as shown in FIG. 15. The user would then turn the knob 25 counter-clockwise as indicated by the arrow in FIG. 4. Because the delivery needle 28 is spring loaded to retract, the turning of the knob enables the latch head 24 to move out of the second recess 30 and into an appropriately sized slot 33 in the knob shaft as shown in side view FIG. 4(B). Upon completion of the turning, the latch head rests within the slot 33 on the knob shaft 27 as shown in FIG. 4(F). Once the latch 23 moves into this position, the knob 25 cannot be depressed and the delivery needle 28 cannot extend out of the housing 10 thereby preventing reuse or any potential harm or injury to the user or healthcare provider.

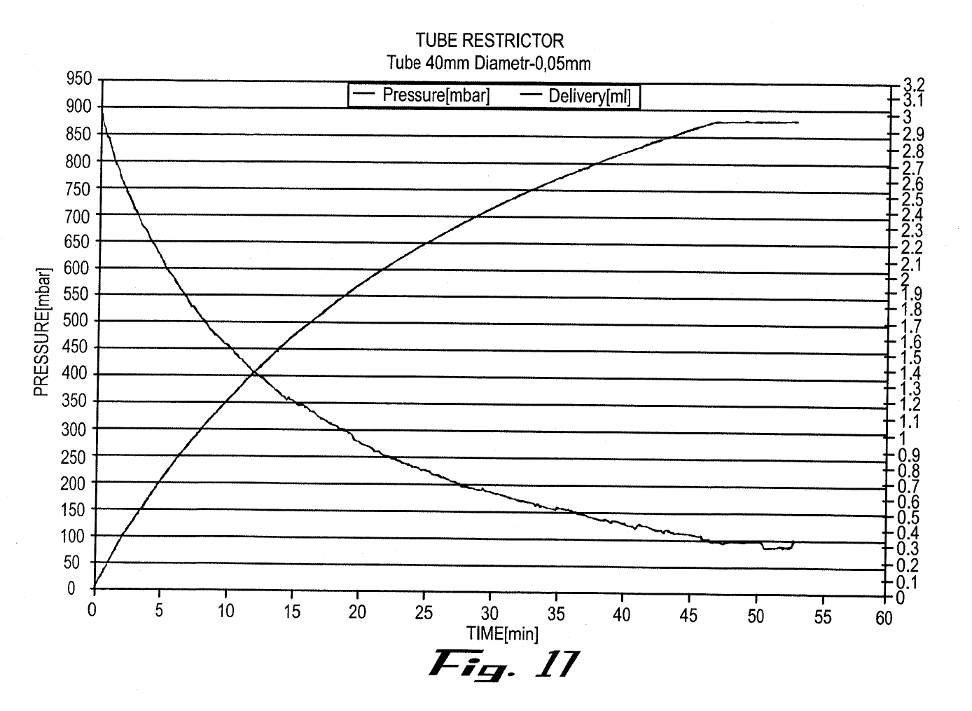

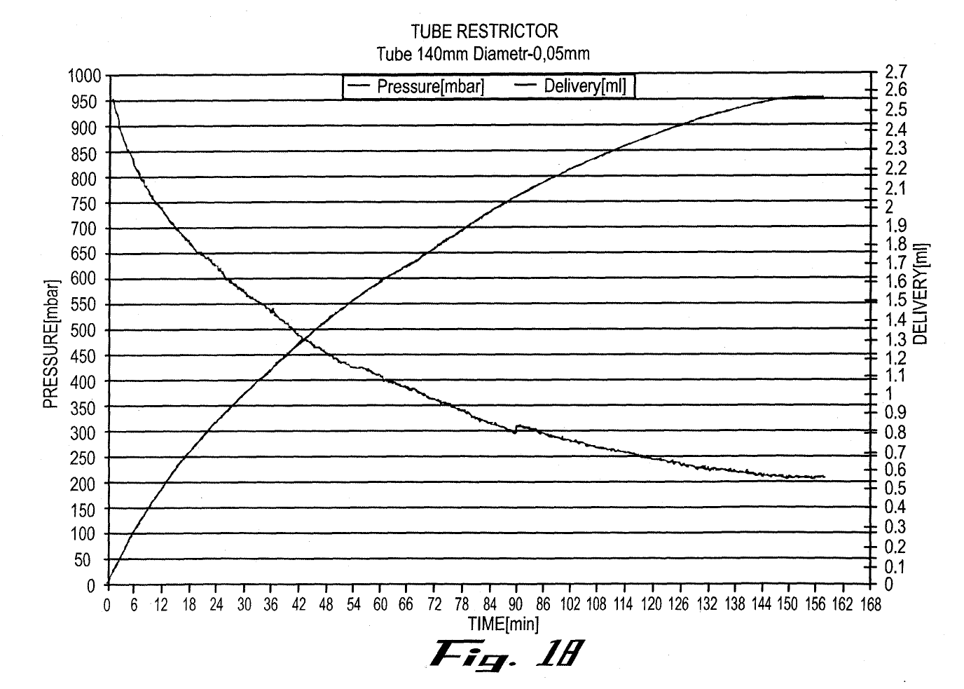

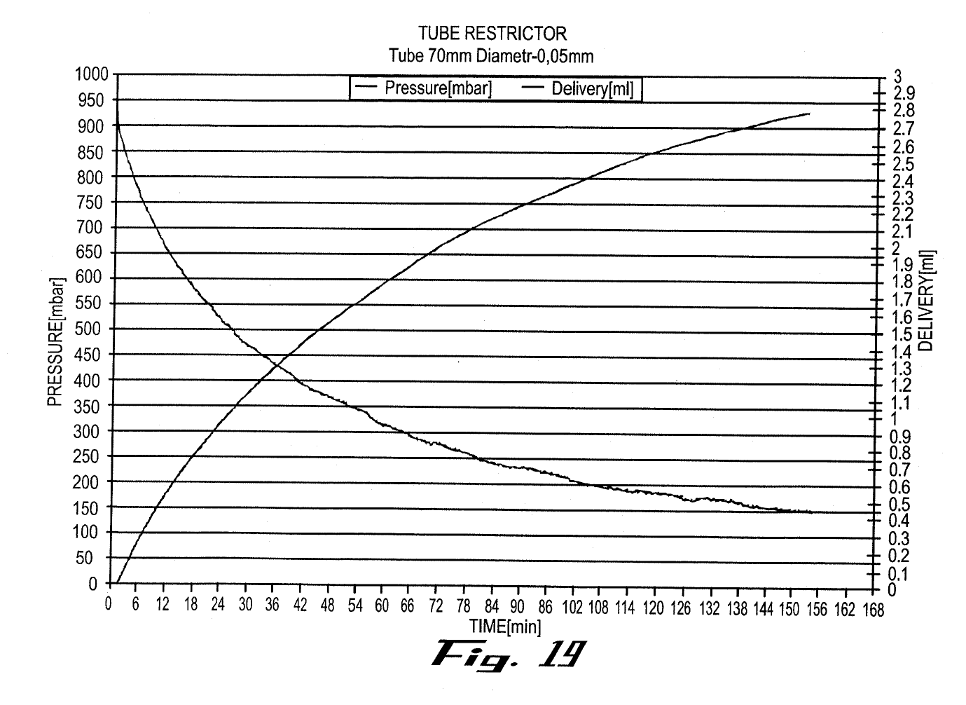

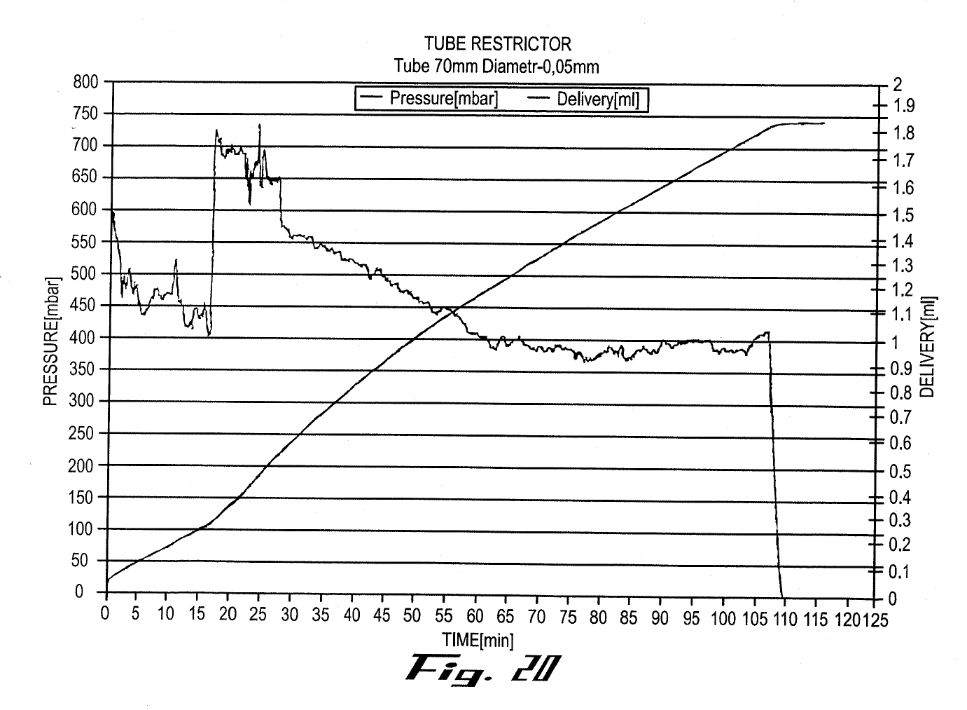

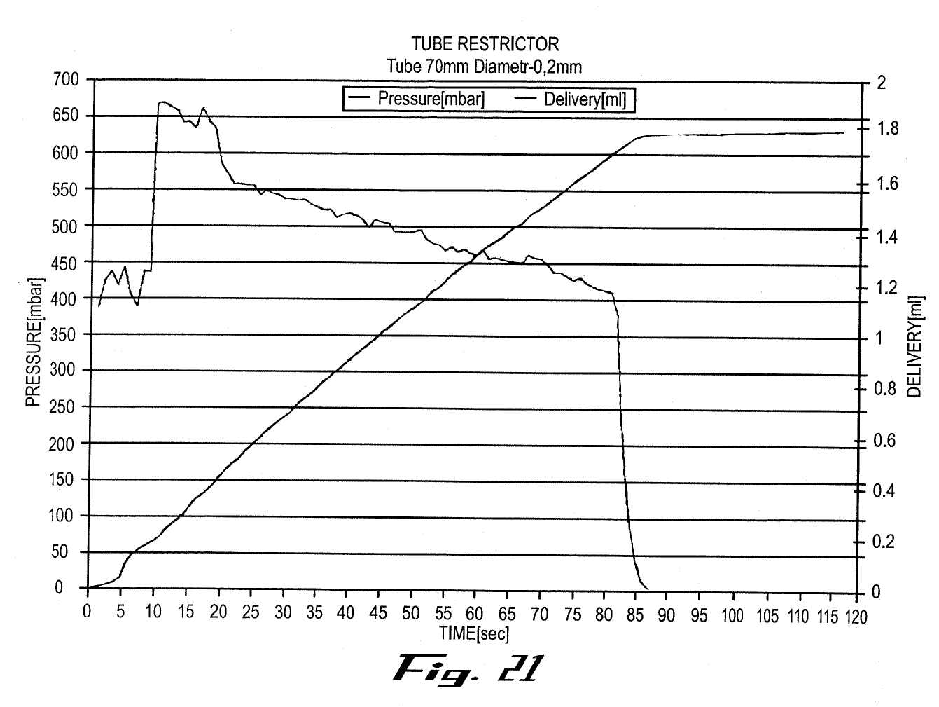

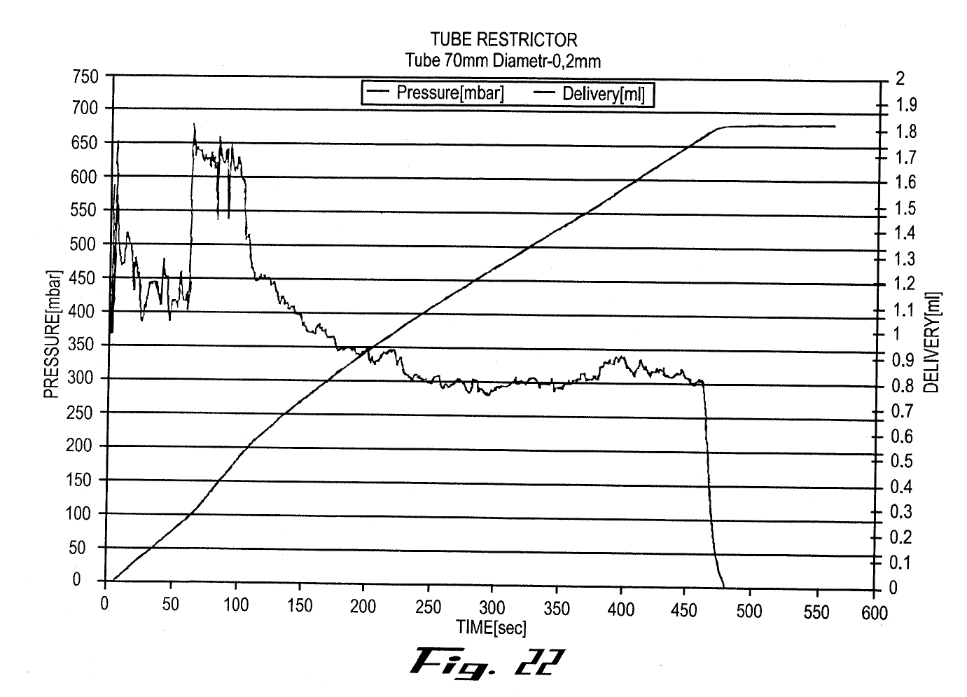

In an effort to determine appropriate flow rate for highly viscous drugs, applicants have completed a number of experiments using various sizes of tubing. The experiments were conducted using a fixed volume of water 50 forced through a length of tubing 52 by means of a spring 54, as shown in FIG. 16. The pressure was measured using a pressure transducer 56. The results of the experiments are set forth in FIGS. 17 through 22. The sizes of the tubing varied in diameter from 0.05 mm to 0.2 mm. The length of tubing used in the experiments was 40 mm, 70 mm and 140 mm.

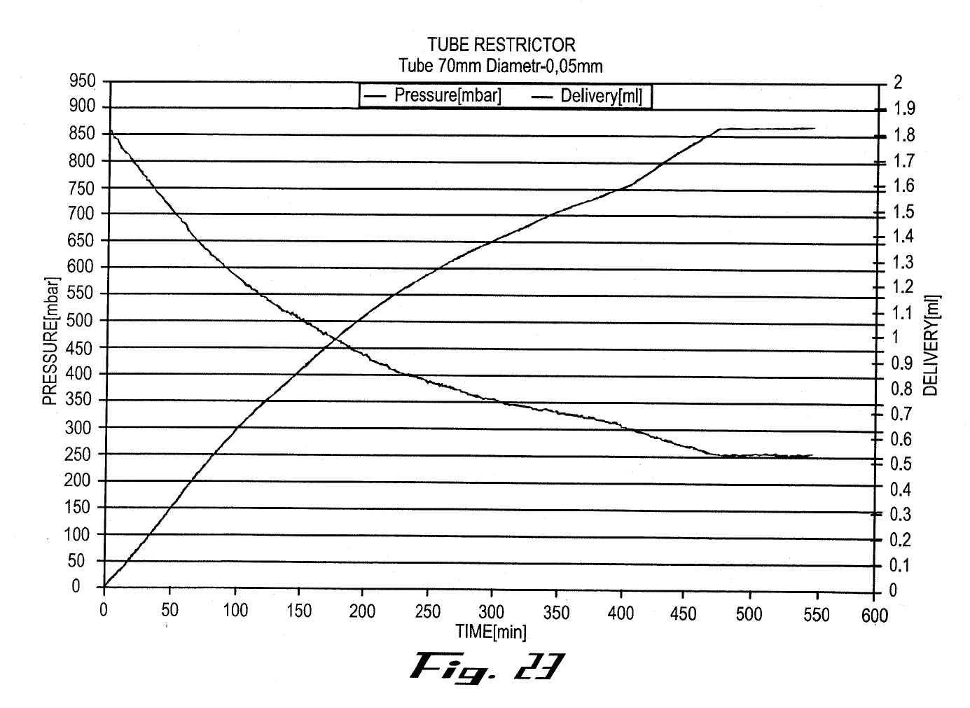

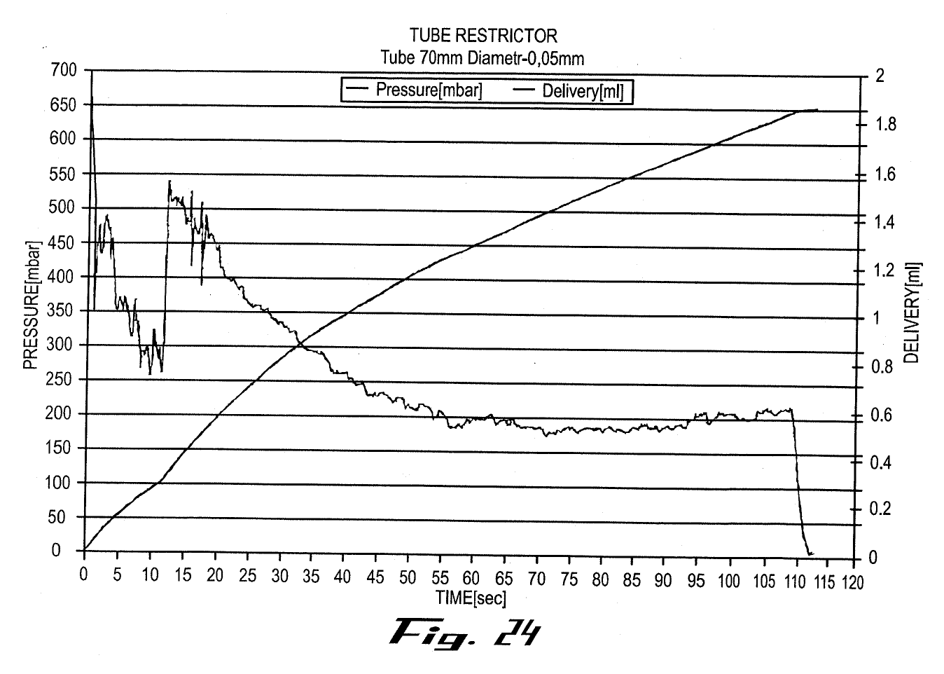

The tubing material is TFL special sub-lite wall tubing manufactured by Zeus. As can be seen from FIGS. 17-24, the delivery rate of drug achieved in the tests was up to 3 ml/hr.

It should be noted that while the experimental tests indicate certain delivery rates, pressure settings and tube dimensions, these parameters may be altered to attain a desired delivery rate or to accommodate a particular drug having a particular viscosity. Moreover, while viscosity of drugs used with the present invention are around 50 cp, the viscosity can be up to 150 cp depending upon the drug's ability to remain in liquid form and not crystalize.

In addition, the volume of drug for delivery can vary depending upon the force applied to move it through the system. In the experiments, the volume of solution ranged from about 2 to 3 milliliters. However, the volume of drug may be altered depending upon the geometry of the flow path and the force used to move drug out of the reservoir.

The system may also include a layer of adhesive applied to the underside of the housing that contacts the skin during delivery. The adhesive is secured to the housing by means of glue or other compound and is covered with a removable liner such as paper. During use, the user removes the liner from the bottom of the housing and applies the housing to the injection site in preparation for delivery. The adhesive secures the housing in position, so that the needle does not move during delivery. Because the delivery time is longer than a standard injection, movement of the needle is to be avoided. Otherwise, any movement of the needle while in the skin can cause the user uneccessary pain and discomfort.

It is further appreciated that the present invention may be used to deliver a number of drugs. The term “drug” used herein includes but is not limited to peptides or proteins (and mimetic thereof), antigens, vaccines, hormones, analgesics, anti-migraine agents, anti-coagulant agents, medications directed to the treatment of diseases and conditions of the central nervous system, narcotic antagonists, immunosuppressants, agents used in the treatment of AIDS, chelating agents, antianginal agents, chemotherapy agents, sedatives, anti-neoplastics, prostaglandins, antidiuretic agents and DNA or DNA/RNA molecules to support gene therapy.

Typical drugs include peptides, proteins or hormones (or any mimetic or analogues or any thereof) such as insulin, calcitonin, calcitonin gene regulating protein, atrial natriuretic protein, colony stimulating factor, betaseron, erythropoietin (EPO), interferons such as α, β or γ interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-1ra; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anticoagulant agents such as heparin, hirudin, and analogues thereof, anti-emetic agents such as scopolamine, ondansetron, domperidone, metoclopramide, and analogues thereof; cardiovacular agents, anti-hypertensive agents and vasodilators such as diltiazem, clonidine, nifedipine, verapamil, isosorbide-5-monotritate, organic nitrates, agents used in treatment of heart disorders, and analogues thereof; sedatives such as benzodiazepines, phenothiazines, and analogues thereof; chelating agents such as defroxanune, and analogues thereof; antidiuretic agents such as desmopressin, vasopressin, and analogues thereof; anti-anginal agents such as fluorouracil, bleomycin, and analogues thereof; anti-neoplastics such as fluorouracil, bleomycin, and analogues thereof; prostaglandins and analogues thereof; and chemotherapy agents such as vincristine, and analogues thereof, treatments for attention deficit disorder, methylphenidate, fluvoxamine, bisoprolol, tacrolimus, sacrolimus and cyclosporin.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A drug delivery system comprising:
a housing;
a drug reservoir received into the housing;
an activation assembly comprising a delivery needle for insertion into the skin of a user upon activation, and an activation device for activating delivery of drug into the skin;
a channel to effect liquid communication between the drug reservoir and delivery needle upon receipt of the drug reservoir into the housing;
a flow restrictor in communication with the moveable channel to restrict the flow of drug therethrough; and
a pump for moving drug out of the drug reservoir, through the delivery needle and into the user when activated.

2. The system of claim 1 wherein the drug reservoir comprises a drug cartridge having a stopper at one end.

3. The system of claim 2 wherein the drug cartridge is received into a recess in the housing.

4. The system of claim 2 wherein the channel comprises a needle capable of piercing the stopper of the drug reservoir.

5. The system of claim 1 wherein the activation device activates liquid communication between the drug reservoir and the delivery needle.

6. The system of claim 1 wherein the activation device activates insertion of the delivery needle into the skin of the user.

7. The system of claim 1 wherein the flow restrictor is a length of flexible tubing.

8. The system of claim 7 wherein the tubing diameter is in the range of between 0.2 and 0.05 mm.

9. The system of claim 1 wherein the delivery needle is a 26 gauge needle.

10. The system of claim 1 wherein the drug has a viscousity of up to 150 cp.

11. The system of claim 7 wherein the tubing length is in the range of between 40 and 140 mm.

12. The system of claim 1 wherein the pump comprises a spring that applies pressure to the drug reservoir thereby forcing drug out of the drug reservoir.

13. The system of claim 10 wherein the pressure applied by the spring is in the range of between 400 and 1000 mbar.

14. The system of claim 7 wherein the tubing is made of plastic.

15. The system of claim 1 wherein the delivery needle moves from a first stored position, to a second delivery position when the delivery needle is moved into the skin of a user, to a third locked position within the housing of the system.

16. A method of delivering drug to a user comprising the steps of:
providing a drug delivery system having a housing, a drug reservoir, an activation assembly, the activation assembly having a delivery needle for insertion into the skin of a user, and an activation device for activating the insertion of the delivery needle into the skin, and a channel to effect liquid communication between the drug reservoir and delivery needle upon activation;
activating the system thereby causing liquid communication between the drug reservoir and the delivery needle;
moving drug out of the drug reservoir, through the delivery needle and into the user; and
restricting the flow of drug along the channel between the drug reservoir and the delivery needle.

17. The method of claim 16 wherein the drug reservoir comprises a drug cartridge having a stopper at one end.

18. The method of claim 17 wherein the step of moving drug out of the reservoir is accomplished by applying force to the stopper.

19. The method of claim 18 wherein the step of applying force is accomplished through the use of a spring.

20. The method of claim 16 wherein the step of restricting the flow is accomplished by causing the drug to flow from the drug reservoir into a length of tubing and into the delivery needle.

21. The method of claim 16 further comprising the step of adhering the system to the user's skin prior to step of activating the system.

22. The device of claim 1 further comprising a layer of adhesive on the bottom of the housing to adhere the housing to the user's skin.
